(19) United States
(12) Patent Application Publication
(10) Pub. No.: US 2025/0340671 A1
(43) Pub. Date: Nov. 6, 2025

(54) METHODS AND PHARMACEUTICAL COMPOSITIONS FOR REDUCING THE NEUROMODULATORY EFFECT OF COCAINE

(71) Applicant: University of Cincinnati, Cincinnati, OH (US)

(72) Inventors: Dakota Zinani, Cincinnati, OH (US); Andrew B. Norman, Cincinnati, OH (US)

(73) Assignee: University of Cincinnati, Cincinnati, OH (US)

(21) Appl. No.: 18/869,919

(22) PCT Filed: Jun. 1, 2023
(86) PCT No.: PCT/US2023/024166

§ 371 Date: Nov. 27, 2024

Related U.S. Application Data
(60) Provisional application No. 63/347,770, filed on Jun. 1, 2022.

(51) Int. Cl. C07K 16/44; A61K 31/55; A61K 39/00; A61P 25/30

(52) U.S. Cl. CPC C07K 16/44; A61K 31/55; A61P 25/30; A61K 2039/505; A61K 2039/545

(57) ABSTRACT

A method of reducing the risk of a cocaine-addicted subject relapsing in addiction is provided, the method including administering to the subject a combination of an effective amount of humanized 2E2 (h2E2) monoclonal antibody and an effective amount of a dopamine 1 receptor antagonist, such as SCH23390. Also provided are methods of reducing cocaine drug-seeking behavior, methods of reducing the neuromodulatory effect of cocaine on the brain of a subject, and a pharmaceutical composition including h2E2 monoclonal antibody, a dopamine 1 receptor antagonist, and a pharmaceutically-acceptable excipient.

### CROSS REFERENCE TO RELATED APPLICATION

This application claims priority to U.S. Provisional Application Ser. No. 63/347,770, filed Jun. 1, 2022, the entire contents of which are incorporated herein by reference.

### STATEMENT REGARDING FEDERALLY SPONSORED RESEARCH OR DEVELOPMENT

This invention was made with government support under DA050330 awarded by the National Institutes of Health. The government has certain rights in the invention.

### TECHNICAL FIELD

The present disclosure relates to the field of cocaine use disorders. Specifically, this disclosure relates to methods and pharmaceutical compositions for ameliorating or treating cocaine use disorders, including addiction and relapse.

### BACKGROUND

Cocaine related deaths have more than quadrupled in recent years, going from 5,415deaths in 2014 to 24,486 deaths in 2021. Additionally, an estimated 1.4 million Americans have had a cocaine use disorder in the past 12 months. Repeated cocaine use at increasingly high doses can lead to increased irritability, restlessness, panic attacks, paranoia, and even psychosis. Cocaine use can also lead to organ damage and put users at increased risk for strokes, seizures, and other neurological problems.

Currently, there are no FDA approved medications for treating cocaine use disorders. A need exists for additional therapeutic approaches for ameliorating and/or treating cocaine use disorders, including addiction and relapse.

### SUMMARY

Accordingly, provided herein are methods and compositions for use in treating cocaine use disorders. The methods and compositions disclosed herein comprise a combination of humanized 2E2 monoclonal (h2E2) antibody and a dopamine 1 receptor antagonist which work together to reduce the neuromodulatory effect of cocaine and reduce drug-seeking behavior. particularly after single-dose reinstatement.

In one embodiment, a method of reducing the risk of a cocaine-addicted subject relapsing in addiction is provided, the method comprising administering to the subject a combination comprising an effective amount of h2E2 monoclonal antibody and an effective amount of a dopamine 1 receptor antagonist.

In another embodiment, a method of reducing cocaine drug-seeking behavior in a subject in need thereof is provided, the method comprising administering to the subject a combination comprising an effective amount of h2E2 monoclonal antibody and an effective amount of a dopamine 1 receptor antagonist.

In another embodiment, a method of reducing the neuromodulatory effect of cocaine on a brain of a subject in need thereof is provided. The method includes administering to the subject a combination comprising an effective amount of h2E2 monoclonal antibody and an effective amount of a dopamine 1 receptor antagonist.

In another embodiment, a pharmaceutical composition is provided, the pharmaceutical composition comprising an effective amount of h2E2 monoclonal antibody, an effective amount of a dopamine 1 receptor antagonist, and a pharmaceutically-acceptable excipient.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

FIG. 1 is a graph illustrating results of three different representative sessions with the same rat showing the cumulative number of lever-presses and duration of lever-pressing behavior following cocaine single dose reinstatement for control (●), SCH23390 10 nmol/kg (□), and SCH23390 30 nmol/kg (▴).

FIG. 2 is a graph illustrating results of three different representative sessions with the same rat showing the calculated cocaine level at the time of each corresponding lever-press of FIG. 1, following cocaine single dose reinstatement for control (●), SCH23390 10 nmol/kg (□), and SCH23390 30 nmol/kg (▴).

FIG. 3 is a graph illustrating results of two representative sessions with the same rat on two different days, showing the cumulative number of lever presses and duration of lever-pressing following cocaine single dose reinstatement for control (○) and h2E2 treatment (●).

FIG. 4 is a graph illustrating results of two different representative sessions with the same rat, showing the calculated cocaine level at the time of each corresponding lever press from FIG. 3. following cocaine single dose reinstatement for control (○) and h2E2 treatment (●).

FIG. 5 is a graph of the mean latency to lever-pressing compared to baseline (○) and after administration of h2E2 (●).

FIG. 6 is a graph illustrating the latency of lever-pressing activity after single dose reinstatement in response to each treatment of the dopamine 1 receptor antagonist SCH23390.

FIG. 7 is a graph showing that administration of SCH23390 decreases the duration of lever-pressing behavior.

FIG. 8 is a graph showing mean latency times to lever-pressing activity for each day after administration of h2E2 (●) and a combination of h2E2 and the dopamine 1 receptor antagonist SCH23390 (▪).

FIG. 9 is a graph showing mean latency times to activity after administration of h2E2 (●) and both h2E2 and the dopamine 1 receptor antagonist SCH23390 (▪), over the course of four days following dopamine 1 receptor antagonist SCH23390 in the experimental group.

FIG. 10 is a graph showing mean duration of lever-pressing for each day after administration of h2E2 (●) and both h2E2 and the dopamine 1 receptor antagonist SCH23390 (▪).

FIG. 11 is a graph illustrating the effect of each SCH23390 dose (control, 10 nmol/kg, 20 nmol/kg, 30 nmol/kg) on the duration of lever-pressing behavior on the first day of administration of SCH23390.

### DETAILED DESCRIPTION

The details of embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

“Cocaine use disorders,” as used herein, include cocaine dependence, addiction, overdose, and/or relapse, and any other disorder resulting in whole or in part from cocaine use by a subject.

“Humanized 2E2 monoclonal antibody,” “h2E2 monoclonal antibody,” and “h2E2 mAb,” as used herein, refer to a humanized chimeric human/murine anti-cocaine mAb 2E2. h2E2 is described and characterized in U.S. Pat. No. 9,957,332, which is incorporated herein by reference in its entirety.

“Dopamine 1 receptor” or “D1 receptor,” as used herein, refer to a protein receptor encoded by the DRD1 gene in humans. D1 receptors are the most abundant dopamine receptors in the central nervous system. D1 receptors regulate the memory, learning, and growth of neurons and also participate in reward systems and locomotor activity. D1 receptors are involved in drug addiction and facilitate gene expression changes that occur during addiction.

Dopamine is a modulator of neuronal activity and synaptic plasticity throughout the central nervous system. The actions of dopamine are mediated by dopamine receptors, which are G protein-coupled receptors that are implicated in many neurological processes, including motivational and incentive salience, cognition, memory, learning, and fine motor control, as well as modulation of neuroendrocrine signaling. Abnormal dopamine receptor signaling is associated with various neuropsychiatric disorders. Antipsychotic drugs are typically dopamine receptor antagonists. There are at least 5 subtypes of dopamine antagonists, dopamine 1 (D1), dopamine 2 (D2), dopamine 3 (D3), dopamine 4 (D4), and dopamine 5 (D5). The D1 and D5 receptors are members of the D1-like family, while the D2, D3, and D4 receptors are members of the D2-like family.

Dopamine 1 receptor antagonists, or D1 receptor agonists, are molecules that binds the D1 receptor, belonging to the D1-like family of dopamine receptors, and at least partially block the activity thereof. Suitable dopamine 1 receptor antagonists for use in the disclosed methods and compositions include, but are not limited to, SCH23390, asenapine, ecopipam, NSC 170976, L-tetrahydropalmatine, R6238, pimethexene maleate, NNC 687, NNC 756, and the like. In specific embodiments, the dopamine 1 receptor antagonist is SCH23390. In another specific embodiment, the dopamine 1 receptor antagonist crosses the blood brain barrier of a subject.

Dopamine 5 receptor antagonists, or D5 receptor agonists, are molecules that binds the D5 receptor, belonging to the D1-like family of dopamine receptors, and at least partially block the activity thereof. Suitable dopamine 5 receptor antagonists for use in the disclosed methods and compositions include, but are not limited to, 4-chloro-7-methyl-5,6,7,8,9,14-hexahydrodibenz[d,g]azecin-3-ol, molindone, SKF28293, chlorprothixene, olanzapine, and the like.

Dopamine 2 receptor antagonists, or D2 receptor agonists, are molecules that binds the D2 receptor, belonging to the D2-like family of dopamine receptors, and at least partially block the activity thereof. Suitable dopamine 2 receptor antagonists for use in the disclosed methods and compositions include, but are not limited to, cinnarizine, chloroethylnorapomorphine, desmethoxyfallypride, domperidone, metoclopramide, eticlopride, fallypride, hydroxyzine, itopride, L-741,626, SV293, yohimbine, buspirone, haloperidol, thioridazine, triflupromazine, olanzapine, aripirazole, molindone, spiperone, nemonpride, 3-ppp, aceprometazine, amisulpride, aripiprazole, bl-1020, blonanserin, chlorprothixene, chlorpromazine, deudomperidone, doxepin, flunarizine, imipramine, ketanserin, 1-741626, loxapine, lurasidone, melitracen, metopimazine, ocaperidone, opipramol, paliperidone, panamesine, perospirone, phenothiazine, pimozide, pipamperone, prochlorperazine dimaleate, pridopidine, promazine, quetiapine, raclopride, sarizotan, stepholidine, sulpiride, tiapride, terguride, tetrabenazine, tetrahydroberberine, tetrahydropalmatine, tiotixene, trazpiroben, trimethobenzamide, and the like.

Dopamine 3 receptor antagonists, or D3 receptor agonists, are molecules that binds the D3 receptor, belonging to the D2-like family of dopamine receptors, and at least partially block the activity thereof. Suitable dopamine 3 receptor antagonists for use in the disclosed methods and compositions include, but are not limited to, amisulpride, buspirone, cyproheptadine, PG 01037, domperidone, FAUC 365, GR-103,691, GSK598809, haloperidol, nafadotride, NGB-2904, PNU-99,194, raclopride, S-14,297, S33084, SB-277011-A, SR 27502, sulpiride, U99194, YQA14, risperidone, aripiprazole, blonanserin, chlorprothixene, deudomperidone, loxapine, metopimazine, olanzapine, panamesine, pimozide, pipamperone, sb-277,011-a, tiapride, tiotixene, trazpiroben, and the like.

Dopamine 4 receptor antagonists, or D4 receptor agonists, are molecules that binds the D4 receptor, belonging to the D2-like family of dopamine receptors, and at least partially block the activity thereof. Suitable dopamine 4 receptor antagonists for use in the disclosed methods and compositions include, but are not limited to, A-381393, FAUC 213, L-745,870, L-750,667, ML-398, S 18126, fananserin, olanzapine, buspirone, chlorprothixene, 1-745,870, a-381393, pimozide, pipamperone, promazine, terguride, and the like.

It should be appreciated that any dopamine receptor antagonist that crosses the blood brain barrier of a subject is suitable for use in the methods and compositions of the present disclosure. Because h2E2 binds cocaine directly and does not interfere with binding of antagonists to dopamine receptors, both D1-like and D2-like receptor antagonists are suitable for use in combination with h2E2.

As used herein, the term “subject” generally refers to a living being (e.g., animal or human) capable of suffering from glioblastoma multiforme. In a specific embodiment, the subject is a mammal, such as a human, rat, mouse, monkey, horse, cow, pig, dog, cat, guinea pig, etc. In a more specific embodiment, the subject is a human subject, a rat, or a mouse. In a more specific embodiment, the subject is a human.

The terms “treat,” “treatment,” and “treating,” as used herein, refer to a method of alleviating or abrogating a disease, disorder, and/or symptoms thereof. In a specific embodiment, the disease or disorder is a cocaine use disorder. In a very specific embodiment, the disorder is cocaine dependence, addiction, overdose, and/or relapse.

As used herein, the terms “administer” or “administration” may comprise administration routes such as enteral (e.g., oral, sublingual, buccal, or rectal), parenteral (e.g., intravenous, intramuscular, subcutaneous, intraarterial, intrathecal), intranasal, inhaled, vaginal, transdermal, etc., so long as the route of administration results in reducing the neuromodulatory effect of cocaine on the brain of the subject, reducing cocaine drug-seeking behavior, or reduces the risk of relapse in a cocaine-addicted subject. In specific embodiments, the administration route is intravenous. In embodiments, each of h2E2 and the dopamine receptor 1 antagonist may be administered intravenously by injection or infusion.

“Co-administered,” as used herein, refers to administration of h2E2 and a dopamine receptor 1 antagonist such that both agents can simultaneously achieve a physiological effect, e.g., in a recipient subject. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the subject. Thus, in embodiments, the h2E2 and the dopamine receptor 1 antagonist may be administered concurrently or sequentially.

“Effective amount,” as used herein, refers to an amount of an agent sufficient to achieve a desired biological effect. Effective amounts will vary based on a subject's age, body weight, condition, and the like, and may be determined by one of skill in the art in view of the present disclosure. The compositions of the present disclosure can be administered by either single or multiple dosages of an effective amount. In embodiments, the effective amount of an agent is an amount sufficient to treat a cocaine use disorder. In specific embodiments, the effective amount is an amount sufficient to reduce the risk of relapse in a cocaine-addicted subject, to reduce the risk of cocaine drug-seeking in a subject in need thereof, and/or to reduce the neuromodulatory effect of cocaine on the brain of the subject.

“Cocaine priming threshold,” as used herein, refers to the minimum amount (cumulative concentration or dose) of cocaine that reinstates lever-pressing behavior and/or self-administration behavior of cocaine in a subject.

“Cocaine satiety threshold,” as used herein, refers to the maximum amount (cumulative concentration or dose) of cocaine in the body that induces lever-pressing behavior and/or self-administration behavior. At cocaine levels above the satiety threshold the probability of lever-pressing or self-administration behavior is low.

“Cocaine compulsion zone,” as used herein, refers to the range of cocaine levels in the body or brain that induce lever-pressing behavior and/or self-administration behavior. The lower limit of the cocaine compulsion zone is the cocaine priming threshold and the upper limit of the cocaine compulsion zone is the cocaine satiety threshold. As dopamine receptor antagonists raise both the priming and satiety thresholds, the result of administration of a dopamine receptor antagonist is that the cocaine compulsion zone is raised.

Abstinent cocaine users often report that taking a small amount of cocaine can lead to a full relapse of addiction and drug-seeking behavior. This “priming” effect has been studied in both humans and animals trained to self-administer cocaine. Single dose reinstatement (SDR) primes rats by administering a specific dose of cocaine and has traditionally been used to study cocaine-induced relapse behavior. The compulsion zone theory, when applied to SDR, has shown the latency time until drug seeking activity begins, as well as the duration of activity, follow pharmacokinetic (PK) parameters of cocaine. Therefore, SDR is an accurate model for studying cocaine induced self-administering behavior.

The present disclosure is directed to the effects of humanized anti-cocaine monoclonal antibody (h2E2) on lever-pressing behavior in rats following a single dose reinstatement event. h2E2 binds to cocaine with 3.9 nM affinity and sequesters it from plasma, effectively blocking cocaine from crossing the blood-brain barrier. Once bound, cocaine is made pharmacologically inert and its elimination half-life is extended, thereby reducing the clearance of the cocaine.

In rats, the number and rate of lever-pressing behavior following termination of access to cocaine has been shown to be a conditioned response, as rats on a progressive ratio schedule of self-administration showed a much higher rate and number of presses compared to rats on fixed ratio 1 schedules. While the rate and number of lever-presses were shown to be an unreliable metric, the duration of the lever-pressing behavior was constant, consistent with the compulsion zone theory.

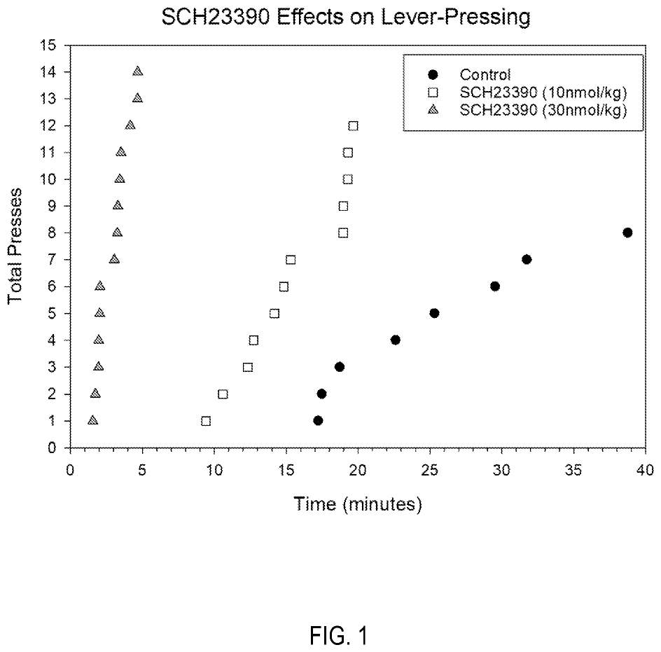

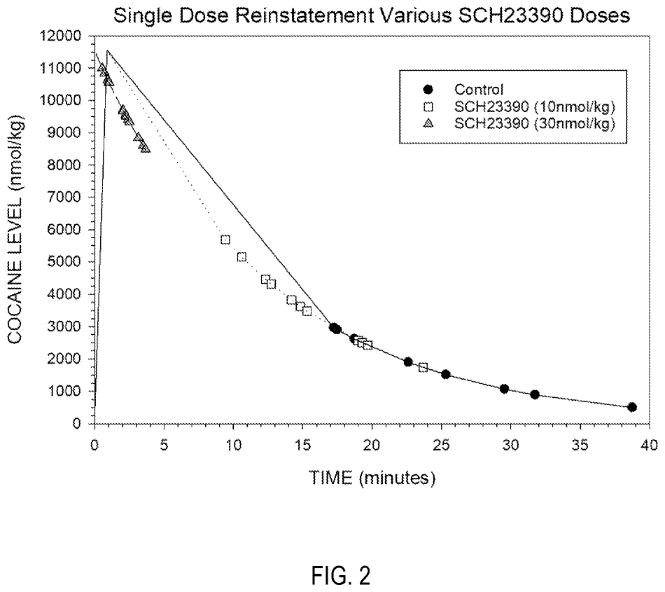

The pharmacokinetic and pharmacodynamic properties of cocaine that govern self-administration behavior may be altered by the introduction of h2E2 and dopamine 1 receptor antagonists. For example, as shown in FIG. 1, the duration of lever presses in a rat subjected to SDR is shortened by the presence of dopamine 1 receptor antagonist SCH23390. Correspondingly, as shown in FIG. 2, the calculated cocaine level at the moment of each of the lever-presses of FIG. 1 shows that the rats begin cocaine induced lever-pressing behavior at higher calculated cocaine levels when the dopamine 1 receptor antagonist SCH23390 is administered. Thus, administering the dopamine 1 receptor antagonist SCH23390 caused a shorter lever-pressing behavior latency time, a higher calculated cocaine level at the onset of lever-pressing activity, and an shorter overall duration of lever-pressing.

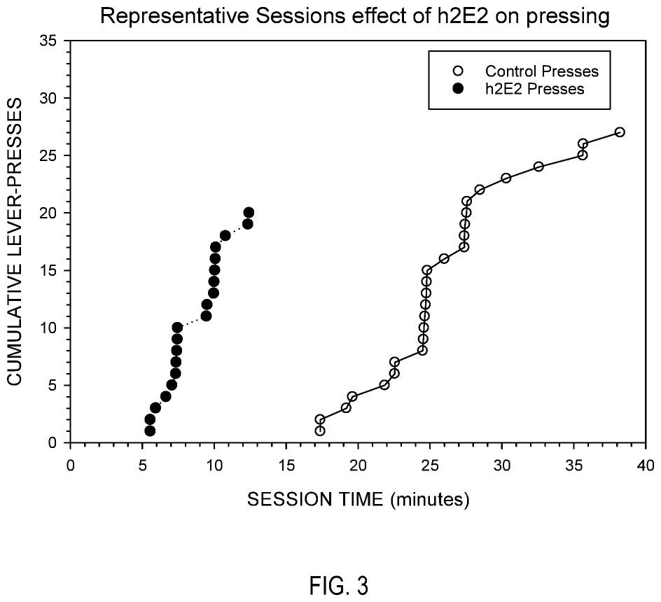

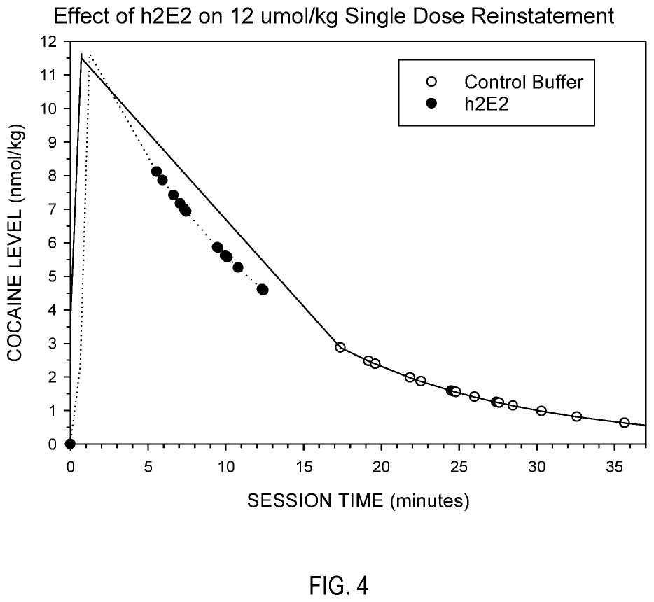

Similar to the results obtained with dopamine 1 receptor antagonist SCH23390, both the latency and duration of lever-pressing activity was significantly decreased following administration of h2E2 in rats one hour before SDR, as shown in FIG. 3. Additionally, as with dopamine 1 receptor antagonist SCH23390, the administration of h2E2 correlated to lever-pressing behavior both beginning and ending earlier when compared to the control buffer, as also shown in FIG. 3. The levels of cocaine at the moment of each lever-press and the level of cocaine at the onset of lever-pressing activity is increased above the control buffer when h2E2 is administered, as shown in FIG. 4. However, the effects of h2E2 began to wean down in accordance with the rate of clearance or half-life of h2E2 antibody, which is about 7 days in rats. In rats, the effects were gone following 21 days.

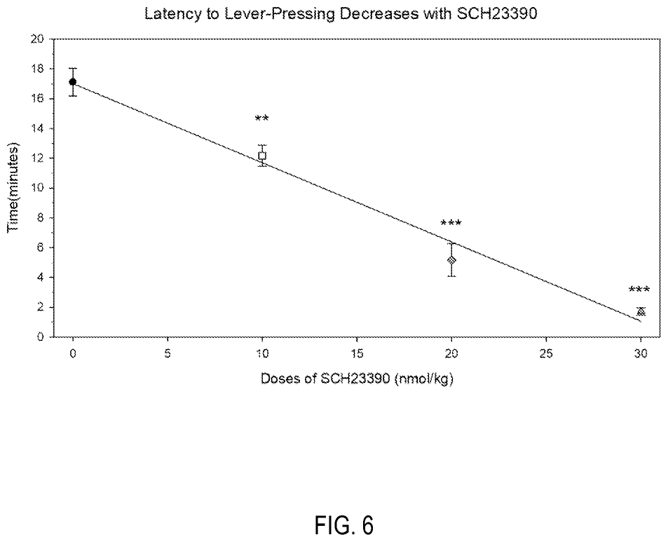

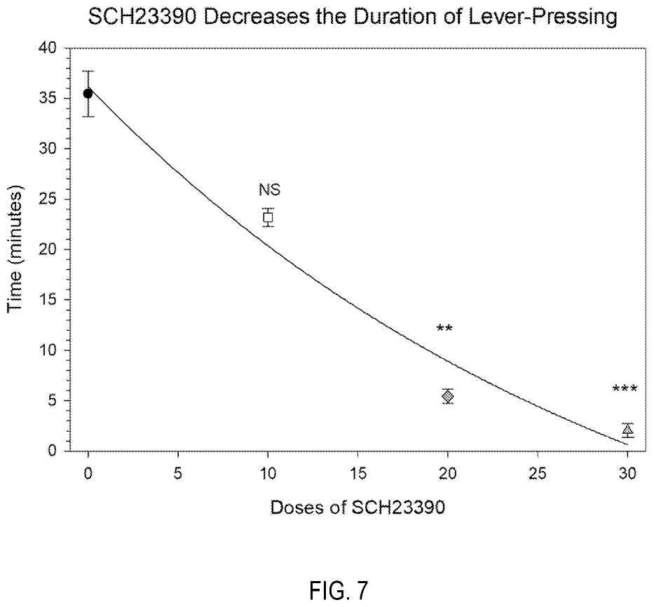

The pharmacodynamic principles of the compulsion zone hold true following a single cocaine dose reinstatement, with prior administration at doses of dopamine 1 receptor antagonist SCH23390, resulting in the cocaine level at which lever-pressing activity commences increasing in a dose dependent manner (FIG. 1 and FIG. 2). These pharmacodynamic changes shift the cocaine levels required to induce lever-pressing behavior, leading to an overall decrease in the latency of lever-pressing (FIG. 6) and the duration of lever-pressing (FIG. 7). Thus, dopamine 1 receptor antagonists such as SCH23390 are capable of improving the efficacy of h2E2 as it is being eliminated from the body. Therefore, dopamine 1 receptor antagonists such as SCH23390 are an effective add-on to treatment to h2E2 treatment.

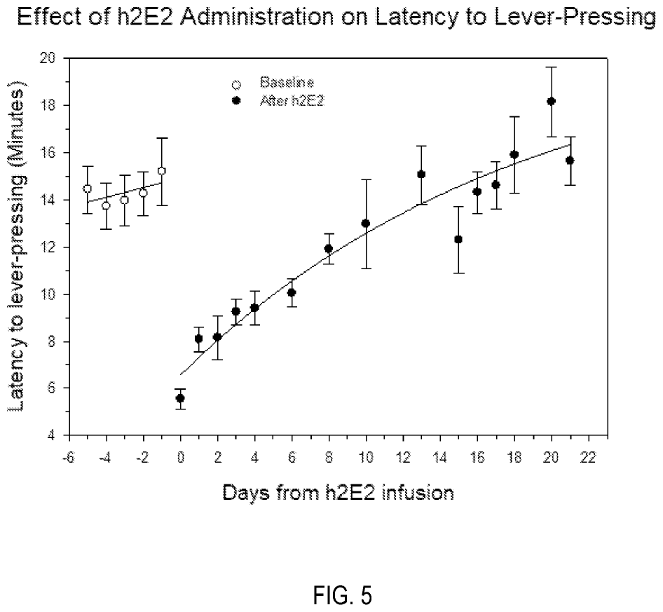

The pharmacokinetic principles of the compulsion zone also hold true for the administration of the humanized anti-cocaine monoclonal antibody following SDR. Following SDR, an overall decrease in latency of lever-pressing in the presence of h2E2 is initially observed, followed by a gradual increase as the antibody is eliminated from the body, as shown in FIG. 5.

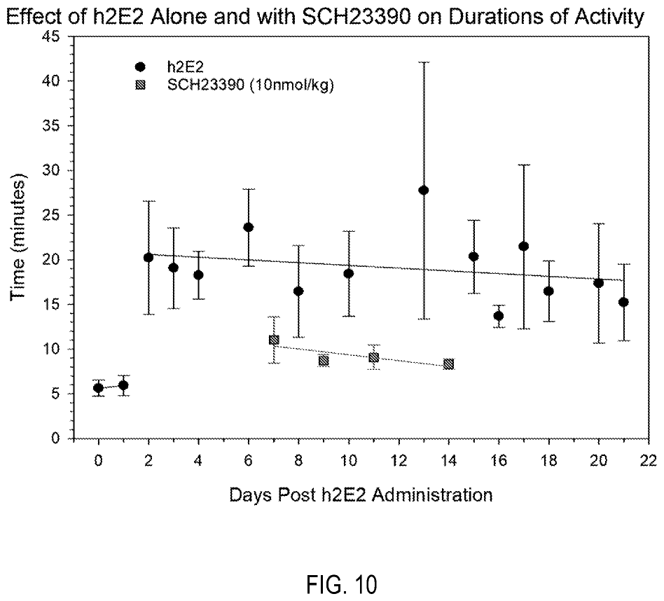

The presence of h2E2 following SDR also corresponded to a decreased duration of the lever-pressing activity for the two days immediately following administration (FIG. 10). However, after two days the durations sharply increased and remained relatively constant, as shown in FIG. 10. Without being bound by theory, it is believed that this trend is observed because in the first several days after the infusion of h2E2, the antibody levels are at their highest, the majority of the cocaine is bound by the antibody, and the amount of cocaine delivered to brain is the lowest. As such, the amount of cocaine reaching the brain immediately following the administration of h2E2 is the lowest concentration and could be considered a low dose that starts the subject within the compulsion zone following priming. This leads to an overall shorter latency, and duration of lever-pressing behavior, as it takes less time for the cocaine levels to fall below the compulsion zone.

However, as h2E2 is eliminated from the body over the course of days, the concentration of cocaine penetrating the blood brain barrier increases until the amount surpasses the compulsion zone. These findings further support the application of therapeutics administered following SDR as a way to reduce the neuromodulatory effect of cocaine on a brain of a subject in need thereof, to reduce the risk of a cocaine-addicted subject relapsing in addiction, and to reduce cocaine drug-seeking behavior in a subject in need thereof.

When used in combination, h2E2 sequesters equimolar amounts of cocaine in the blood plasma, thereby preventing the cocaine from crossing blood-brain barrier, while the dopamine 1 receptor antagonist binds to dopamine 1 receptor in the subject's brain, thereby altering the boundaries of the cocaine compulsion zone. Thus, without being bound by theory, h2E2 and dopamine 1 receptor antagonist are respectively believed to block two independent cocaine induced mechanisms. Additionally, when dopamine 1 receptor antagonist SCH23390 was used in combination with h2E2, a portion of the dopamine 1 receptor antagonist's initial effect on reducing latency and duration of activity was restored.

The data presented herein support the use of combinatorial therapy to provide both a long-term safety net for subjects with cocaine use disorders through the administration of an effective amount of h2E2 and an in-the-moment reducer of cocaine-induced drug seeking behavior through administration of an effective amount of a dopamine 1 receptor antagonist. Additionally, as the h2E2 wears off, the introduction of a dopamine 1 receptor antagonist is believed to compensate for the loss of efficacy by pharmacodynamically increasing the boundaries of the compulsion zone, thereby reducing the probability of relapse even when h2E2 levels are diminished.

Thus, h2E2 co-administered with a dopamine 1 receptor antagonist such as SCH23390 will at least additively reduce drug seeking behavior in subjects suffering from a cocaine relapse. As demonstrated by the rat model, a subject suffering from a cocaine use disorder who has been administered h2E2 (and still has h2E2 in their body), who is also administered a dopamine 1 receptor antagonist will experience a reduction in drug-seeking behavior, as h2E2 prevents cocaine from inducing effects and the dopamine 1 receptor antagonist inhibits any further self-administrations of cocaine from occurring.

In one embodiment, a method of reducing the risk of a cocaine-addicted subject relapsing in addiction is provided, the method comprising administering to the subject a combination comprising: an effective amount of h2E2 monoclonal antibody; and an effective amount of a dopamine 1 receptor antagonist. In some embodiments, the dopamine 1 receptor antagonist is SCH23390. In embodiments, the administration of h2E2 and the dopamine 1 receptor antagonist occurs after the subject receives a priming dose of cocaine after a period of abstinence from cocaine use.

In some embodiments, the h2E2 monoclonal antibody binds cocaine and sequesters the cocaine from blood plasma of the subject, thereby preventing the bound cocaine from crossing the blood-brain barrier; and the dopamine receptor antagonist binds to dopamine receptors in the brain of the subject, reducing cocaine drug-seeking and self-administration behavior in the subject. In some embodiments, the combination raises a subject's cocaine compulsion zone.

In some embodiments, the h2E2 monoclonal antibody and the dopamine 1 receptor antagonist are administered concurrently or sequentially. For example, the h2E2 monoclonal antibody may first be administered to a subject in need thereof, and then the dopamine 1 receptor antagonist may be administered from 10 seconds to 30 days later. For example, the h2E2 monoclonal antibody may first be administered to a subject in need thereof, and then the dopamine 1 receptor antagonist may be administered from 10 seconds to 30 days, from 30 seconds to 30 days, from 1 minute to 30 days, from 5 minutes to 30 days, from 10 minutes to 30 days, from 30 minutes to 30 days, from 1 hour to 30 days, from 2 hours to 30 days, from 4 hours to 30 days, from 6 hours to 30 days, from 12 hours to 30 days, from 1 day to 30 days, from 2 days to 30 days, from 7 days to 30 days, from 14 days to 30 days, from 21 days to 30 days, 10 seconds to 7 days, from 30 seconds to 7 days, from 1 minute to 7 days, from 5 minutes to 7 days, from 10 minutes to 7 days, from 30 minutes to 7 days, from 1 hour to 7 days, from 2 hours to 7 days, from 4 hours to 7 days, from 6 hours to 7 days, from 12 hours to 7 days, from 1 day to 7 days, 10 seconds to 12 hours, from 30 seconds to 12 hours, from 1 minute to 12 hours, from 5 minutes to 12 hours, from 10 minutes to 12 hours, from 30 minutes to 12 hours, from 1 hour to 12 hours, from 2 hours to 12 hours, from 4 hours to 12 hours, or from 6 hours to 12 hours later. Optionally, one or both of the h2E2 monoclonal antibody and the dopamine 1 receptor antagonist may be re-administered to the subject.

In embodiments, the h2E2 monoclonal antibody and the dopamine 1 receptor antagonist exhibit an at least additive effect, and the administration of the agents in combination does not lead to any interference in the activity of each agent. In embodiments, administration of a dopamine 1 receptor antagonist after h2E2 has been administered to a subject serves to increase the efficacy of h2E2 as it is cleared from the body over time.

Administering the h2E2 may occur by any standard administration means. For example, administering the h2E2 may include oral, intravenous, intraarterial, intrathecal, intramuscular, subcutaneous, buccal, sublingual, nasal, inhalation, and transdermal administration, or combinations thereof. Furthermore, administering the dopamine 1 receptor antagonist may occur by any standard administration means. For example, administering the dopamine 1 receptor antagonist may include oral, intravenous, intraarterial, intrathecal, intramuscular, subcutaneous, buccal, sublingual, nasal, inhalation, and transdermal administration, or combinations thereof. In some embodiments, both the h2E2 monoclonal antibody and the dopamine 1 receptor antagonist may be administered by the same administration means. For example, both the h2E2 monoclonal antibody and the dopamine 1 receptor antagonist may be administered using intravenous administration. In some embodiments, the h2E2 monoclonal antibody and the dopamine 1 receptor antagonist may be administered by different administration means. For example, the h2E2monoclonal antibody may be administered via one form of administration and the dopamine 1 receptor antagonist may be administered via another form of administration. In specific embodiments, the h2E2 monoclonal antibody and the dopamine 1 receptor antagonist are administered intravenously, by injection or infusion. In another specific embodiment, the h2E2 monoclonal antibody is administered intravenously and the dopamine receptor antagonist is administered intravenously or via inhalation.

In some embodiments, the h2E2 monoclonal antibody is administered in an amount ranging from about 1 mg/kg to about 500 mg/kg, from about 1 mg/kg to about 400 mg/kg, from about 4 mg/kg to about 500 mg/kg, or from about 4 mg/kg to about 400 mg/kg. In a specific embodiment, the h2E2 mAb is administered in an amount ranging from about 4 mg/kg to about 400 mg/kg.

In some embodiments, the dopamine 1 receptor antagonist is administered in an amount ranging from about 0.1 mg to about 500 mg, from about 0.5 mg to about 500 mg, from about 0.5 mg to about 400 mg, from about 0.5 mg to about 300 mg, from about 0.5 mg to about 200 mg, from about 0.5 mg to about 100 mg, from about 0.5 mg to about 75 mg, or from about 0.5 mg to about 50 mg. In a specific embodiment, the administered dose ranges from about 0.5 mg to about 100 mg.

In another embodiment, a method of reducing cocaine drug-seeking behavior in a subject in need thereof is provided, the method comprising administering to the subject a combination comprising: an effective amount of h2E2 monoclonal antibody; and an effective amount of a dopamine 1 receptor antagonist. In some embodiments, the dopamine 1 receptor antagonist is SCH23390.

In another embodiments, a method of reducing the neuromodulatory effect of cocaine on the brain of a subject in need thereof is provided, the method comprising administering to the subject a combination comprising: an effective amount of h2E2 monoclonal antibody; and an effective amount of a dopamine 1 receptor antagonist. In some embodiments, the dopamine 1 receptor antagonist is SCH23390.

In some embodiments, the subject of the methods provided herein is a mammal, such as a human, rat, mouse, monkey, horse, cow, pig, dog, cat, guinea pig, etc. In a more specific embodiment, the subject is a human subject, a rat, or a mouse. In a more specific embodiment, the subject is a human.

In another embodiment, a pharmaceutical composition is provided, the pharmaceutical composition comprising: an effective amount of h2E2 monoclonal antibody; an effective amount of a dopamine 1 receptor antagonist; and a pharmaceutically-acceptable excipient. In some embodiments, the pharmaceutical composition is formulated for treating or ameliorating cocaine addiction. In some embodiments, the dopamine 1 receptor antagonist is SCH23390.

In some embodiments, pharmaceutical composition may formulated for oral, intravenous, intraarterial, intrathecal, intramuscular, subcutaneous, buccal, sublingual, nasal, inhalation, or transdermal administration. In a specific embodiment, the pharmaceutical composition is formulated for intravenous administration via injection or infusion.

The compositions may be prepared by any methods well known in the art of pharmacy, for example, using methods such as those described in Remington: The Science and Practice of Pharmacy (23rd ed., Adeboye Adejare, ed., 2020, see Section 7: Pharmaceutical Materials and Devices/Industrial Pharmacy). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given pharmaceutical composition.

For parenteral administration, suitable compositions include aqueous and non-aqueous sterile suspensions for intravenous administration. The compositions may be presented in unit dose or multi-dose containers, for example, sealed vials and ampoules.

As will be understood by those of skill in this art, the specific dose level for any particular subject will depend on a variety of factors, including the activity of the agent employed; the age, body weight, general health, and sex of the individual being treated; the time and route of administration; the rate of excretion; and the like.

In embodiments, the pharmaceutical composition may be formulated for injection. In other embodiments, the pharmaceutical composition may be formulated for infusion.

### EXAMPLES

The following examples are given by way of illustration are not intended to limit the scope of the disclosure.

### Example 1. Materials and Methods

### Animals and Catheter Implantations

Male Sprague-Dawley rats (MGI Cat #5651135, RRID: MGI:5651135) weighing between 200 g and 500 g were purchased from Harlan Laboratories (Indianapolis, IN). Rats were housed individually on a 14/10-hour light/dark cycle with unrestricted access to food and water. All studies were conducted in accordance with the National Institutes of Health Guide for the Care and Use of Laboratory Animals and under a protocol approved by the Institutional Animal Care and Use Committee at the University of Cincinnati.

All surgeries were performed using aseptic technique with materials being prior autoclaved or sterilized with vaporized H₂O₂. Rats weighing ˜200 grams were anesthetized with inhaled isoflurane, then the left jugular vein was catheterized with a 3fr polyurethane rounded tip, this catheter was connected to the pin of an Instech Vascular Access Button (VAB), which was tested for patency by flushing sterile saline through before implantation. Three days following implantation surgeries, rats' catheters were flushed with 100 u/ml of heparinized bacteriostatic saline solution to extend the patency of the catheters. Following failure of patency (tested using brevital sodium), this process was repeated on the right jugular vein, the left femoral, and the right femoral vein with each subsequent loss of patency. Following the loss of the fourth vein patency. animals were humanely sacrificed according to IACUC protocols using CO₂ gas, followed by secondary means of euthanasia.

Rats were implanted with indwelling catheters into the right jugular vein under isoflurane anesthesia (RRID:SCR_018956). If re-catheterization was required, catheters were placed in the left jugular and femoral veins as needed throughout the study. Buprenorphine (0.03 mg/rat S.C.) (RRID: AB_10972407) was administered post-surgery for pain control and gentamycin (25 mg/rat S.C.) for three days was used to prevent infection following surgery.

### Cocaine Self-Administration Training

Detailed protocols for cocaine self-administration training can be found in Tsibulsky, V. and Norman, A., Real time computation of in vivo drug levels during drug self-administration experiments, Brain Res. Brain Res. Protoc. 15(1): 38-45 (2005), incorporated herein by reference. In brief, beginning at least 5 days after surgery, rats were trained to self-administer cocaine HCl. Rats were weighed immediately prior to each self-administration session. Self-administration sessions began from 9:00 to 10:00 am. Animals were placed in isolated chambers containing an active and an inactive lever. During training, a unit dose of 3 μmol/kg (1 mg/kg) of cocaine HCl (supplied by the NIDA Drug Supply Program from the Research Triangle Institute) was delivered on a fixed-ratio 1 (FR1) schedule with a 5 s timeout period or equal to the injection time, whichever was longer. A cue light was illuminated for the duration of the timeout. Rats had access to cocaine for 3 h a day, five days a week. The training was considered complete when inter-injection intervals did not systematically deviate from day to day for three consecutive sessions.

### Single-Dose Reinstatement (SDR) of Lever-Pressing

Following training on cocaine self-administration, rats were then switched to a single-dose reinstatement (SDR) session. Rats were run on a self-administration session (as described prior) every Monday to ensure lever-pressing behavior had not been extinguished. SDR sessions were run Tuesday-Friday. After loading the rat into the same chambers, they were administered a single 12 μmol/kg dose of cocaine and their lever-pressing behavior was recorded until 30-minutes of inactivity elapsed. The rats were then returned to their home cages and allowed to rest until the next session.

### Drug Administration, SDR, and Assessment of Lever-Pressing

Four compounds were IV administered to the rats following a single dose of 12 μmol/kg cocaine: saline vehicle, SCH23390 (D1 antagonist), eticlopride (D2 antagonist), and molindone (atypical antipsychotic). Additionally, each antagonist was administered in three different doses of 10 nmol/kg, 20 nmol/kg, 30 nmol/kg for both D1 and D2 antagonists SCH23390 and eticlopride, and .75 μmol/kg, 1.5 μmol/kg, and 3 μmol/kg for the atypical antipsychotic molindone. Following the single bolus infusion of saline or antagonist, rats were placed in the self-administration chambers and administered the single dose of cocaine, during which they had access to both the inactive and active levers. All lever-pressing behavior following the infusion was recorded, the parameters of focus were the total number of active lever-presses as inactive presses were negligible, the latency time to the onset of activity, the duration of activity from first press to last, the calculated cocaine level at the onset of activity (the satiety threshold), and the calculated cocaine level at the cessation of activity (the remission threshold). This process was repeated for each antagonist and each dose.

h2E2 Administration, SDR, and Assessment of Lever-Pressing

Rats established a baseline behavior several days before the administration of h2E2. Rats were then given the 10% sucrose histidine buffer h2E2 vehicle at an equivalent volume as the dose of h2E2 at a rate of 1 ml/kg/hr, then allowed to rest in their home cage for 1 hr as they would for h2E2, following which they were given a single dose of 12 μmol/kg cocaine HCl solution via IV. Rats were administered a dose of 360 μmol/kg dose of h2E2, then returned to their home cage where they stayed for one hr. They were then placed into self-administration chambers where they were given a single dose of 12 μmol/kg cocaine HCl solution via IV. Lever-pressing behavior was recorded until 30 consecutive minutes of no lever-pressing occurred.

SCH23390 Administration after Administration of h2E2

Seven days after administration of h2E2, a 10 nmol/kg dose of SCH23390 was given before the beginning of the 12 μmol/kg cocaine SDR session began. The dose was administered every other day for four sessions with the fourth having a break of two days due to their assessment day. On the seventh day, a 10 nmol/kg dose of SCH23390 was administered before beginning the single dose reinstatement session and a decrease in both the latency by an average of 2.56 minutes and an average duration of 9.28 minutes activity was observed respectively. While these are not equivalent to the effect of h2E2 on day 0, it is believed that a greater dose will yield an increased magnitude of effect.

### Statistical Analysis

The following parameters were recorded during the unloading phase: total number of presses, latency time between the single dose of cocaine and the first lever-press, duration of lever-pressing activity, calculated cocaine level at the onset of activity, and calculated cocaine level at the cessation of lever-pressing activity (also known as the remission threshold). These data were collected from multiple rats over multiple sessions, however, as rats lost catheter patency the overall number of animals used fluctuated. All the parameters for both groups were collected from MedPC then transferred to Sigmaplot 14.5 where an ANOVA was performed on each group and doses, statistical significance was determined by comparing the values to the control saline. Following administration of h2E2 the same parameters were collected each day for 21 days, and statistical significance of these values were compared via RMANOVA in Sigmaplot to the buffer control day −1. Statistical analysis for unloading had the following number of animals (N) and number of sessions (n) for each group Control N=11 n=50, SCH23390 10/20/30 nmol/kg doses N=9 n=46, N=10 n=60, N=5 n=15.

### Example 2. Dopamine 1 Receptor Antagonist and h2E2 Decrease Latency of Lever-Pressing Behavior Alone and in Combination

Rats administered with control saline or control buffer displayed a normal unloading behavior with the P value of P=.97, in terms of latency or duration of activity. The no treatment, saline control (N=11 n=50) and buffer control had mean latencies of 15.49, 17.11, 15.54 respectively and durations of 21.0, 35.44, 26.11 respectively.

Addition of dopamine 1 receptor antagonist SCH23390 alone decreased the latency of lever-pressing behavior in a dose-dependent manner with the doses of 0, 10, 20, 30 nmol/kg resulting in mean latencies of 17.11, 12.17, 5.17, 1.72, respectively (FIG. 6). The mean durations were 35.44, 23.16, 5.43, 2.05, for each dose 0, 10, 20, 30 nmol/kg of SCH23390 respectively (FIG. 7). The total number of rats (N) and sessions run by those rats (n) for each dose 0, 10, 20, 30 nmol/kg of SCH23390 were N=11 n=32, N=8 n=31, N=11 n=14, N=7 n=7, respectively.

Administration of the humanized monoclonal anti-cocaine antibody h2E2 resulted in a mean decreased latency from 15.2 minutes the day before when buffer was administered, to 5.55 minutes following a 360 umol/kg dose of h2E2, and a mean duration of 5.63 minutes (FIG. 5). Over the course of 7 days, the effect of h2E2 on latency behavior is reduced by about 50% that of the first day to a latency of 10.04 minutes, which is consistent with half-life of h2E2 in rats, N=7. As values changed each day, one session was recorded for each rat daily. However, some days it was not possible to complete all sessions, reducing the N to a minimum of 4 on those days. On the seventh day, a 10 nmol/kg dose of SCH23390 was administered before beginning the single dose reinstatement session and a decrease in both the latency by an average of 2.56 minutes and an average duration of 9.28 minutes activity was observed respectively.

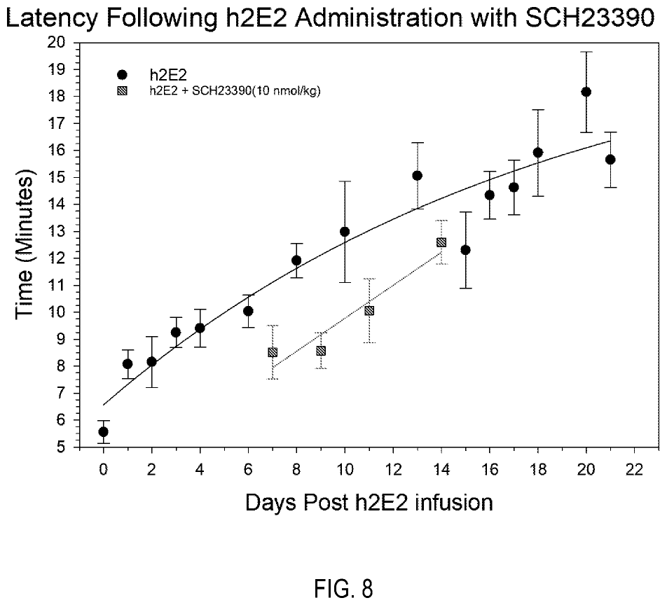

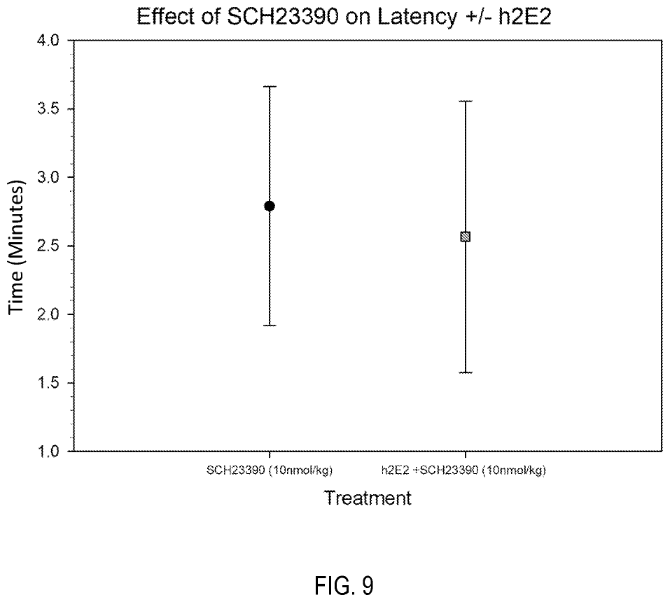

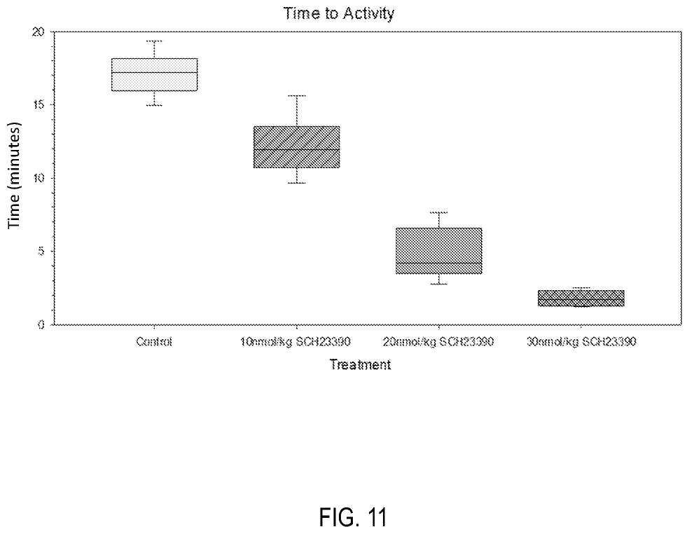

Addition of SCH23390 at the lower dose also resulted in decreased durations of lever-pressing activity to an average of 9.28 minutes, and the average latency of these days was 9.93 minutes (FIG. 8). The addition of low dose SCH23390 alone resulted in an average difference of about 2.81 minutes between vehicle and 10 nmol SCH23390. When administered in the presence of h2E2 the average difference between the tested day and latency of the prior day was 2.56 minutes, which is not a significantly different value h2E2 and SCH23390 (FIG. 9). The first and second days following infusion of h2E2 resulted in an average duration of lever-pressing activity of 5.77 minutes, on par with that of 20 nmol/kg of SCH23390 (FIG. 10). All h2E2-alone days following had a consistent average duration of 19.1 minutes, a value not significantly different from the controls (FIG. 11). On 4 separate days 10 nmol/kg SCH23390 was administered resulting in a decreased duration to 9.28, about half that of days when no SCH23390 was given (FIG. 10).

Seven days after h2E2 was administered, corresponding to the half-life of h2E2 in rats, half of the effect of h2E2 on latency was observed, as shown in FIG. 5. On the eighth day, the lowest effective dose of the dopamine 1 receptor antagonist SCH23390 (10 nmol/kg) was administered every other day, which decreased both the latency and duration of the activity, as shown in FIG. 8.

With h2E2 having a direct impact on the pharmacokinetic effects of cocaine, and dopamine antagonists influencing the pharmacodynamic effects of cocaine following single dose reinstatement, both therapies were tested in a combinatorial fashion. The average latency of a 10 nmol/kg dose of SCH23390 administered prior to a single cocaine dose reinstatement was subtracted from the average vehicle latency, the difference being the average amount of time latency is reduced. This process was repeated for SCH23390 in the presence of h2E2, since the latency with h2E2 changes daily based on the amount of h2E2 present. The SCH23390 dose latencies were subtracted from the prior day when h2E2 was administered alone. If the amount of time the dose (10 nmol/kg) of SCH23390 in the presence of h2E2 was significantly greater than the effect of the same SCH23390 dose in the absence of h2E2 it would indicate a level of synergy. However, the effect of 10 nmol/kg SCH233390 dose on latency in both instances was nearly identical, reducing the latency by 2.81 and 2.56 minutes respectively (FIG. 9). Results show there is no interference of h2E2 with SCH23390.

While the effect was not equivalent to the first day of h2E2 exposure, it did illustrate potentially positive effect of adding SCH23390 together with h2E2 on decreasing the latency and durations of activity following SDR. The anti-cocaine monoclonal antibody h2E2 prevents cocaine from crossing the blood brain barrier, as such it functions as a safety net should an individual relapse. SCH23390 is a small molecule antagonist with a t-max of approximately 30 min that can significantly raise the compulsion zone, reducing the probability of a relapse or decreasing the duration of drug seeking behavior should relapse occur. SCH23390 combined with h2E2 provides a comprehensive treatment utilizing both pharmacokinetic and pharmacodynamic pathways. Due to the extended half-life of h2E2, the mAb functions as a chronic treatment while acute low doses of SCH23390 will reduce the probability of relapse or subsequent drug seeking behavior altogether and decrease the occurrence of negative side effects.

Aspects of the present disclosure can be described with reference to the following numbered clauses, with preferred features laid out in dependent clauses.

1. A method of reducing the risk of a cocaine-addicted subject relapsing in addiction, the method comprising administering to the subject a combination comprising:
an effective amount of h2E2 monoclonal antibody; and
an effective amount of a dopamine 1 receptor antagonist.

2. The method according to clause 1, wherein the dopamine 1 receptor antagonist is SCH23390.

3. The method according to any previous clause, wherein the combination raises the subject's cocaine compulsion zone, thereby reducing drug-seeking behavior of the subject.

4. The method according to any previous clause, wherein the h2E2 monoclonal antibody and the dopamine 1 receptor antagonist are administered concurrently or sequentially.

5. The method according to any previous clause, wherein administering comprises oral. intravenous, intraarterial, intrathecal, intramuscular, subcutaneous, buccal, sublingual, nasal, inhalation, and transdermal administration.

6. The method according to any previous clause, wherein administering comprises intravenous administration of h2E2 and intravenous or inhalation administration of the dopamine 1 receptor antagonist.

7. The method according to any previous clause, wherein the h2E2 monoclonal antibody is administered in an amount from about 4 mg/kg to about 400 mg/kg.

8. The method according to any previous clause, wherein the dopamine 1 receptor antagonist is administered in an amount from about 0.5 mg to about 100 mg.

9. The method according to any previous clause, wherein the subject is a mammal.

10. The method according to any previous clause, wherein the subject is a human, a mouse, or a rat.

11. A method of reducing cocaine drug-seeking behavior in a subject in need thereof, the method comprising administering to the subject a combination comprising:
an effective amount of h2E2 monoclonal antibody; and
an effective amount of a dopamine 1 receptor antagonist.

12. The method according to clause 11, wherein the dopamine 1 receptor antagonist is SCH23390.

13. The method according to any of clauses 11-12, wherein the subject is a mammal.

14. The method according to any of clauses 11-13, wherein the subject is a human, a mouse, or a rat.

15. A method of reducing the neuromodulatory effect of cocaine on a brain of a subject in need thereof, the method comprising administering to the subject a combination comprising:
an effective amount of h2E2 monoclonal antibody; and
an effective amount of a dopamine 1 receptor antagonist.

16. The method according to clause 15, wherein the dopamine 1 receptor antagonist is SCH23390.

17. The method according to any of clauses 15-16, wherein the subject is a mammal.

18. The method according to any of clauses 15-17, wherein the subject is a human, a mouse, or a rat.

19. A pharmaceutical composition comprising:
an effective amount of h2E2 monoclonal antibody;
an effective amount of a dopamine 1 receptor antagonist; and
a pharmaceutically-acceptable excipient.

20. The pharmaceutical composition according to clause 19, wherein the pharmaceutical composition is formulated for treating or ameliorating cocaine addiction.

21. The pharmaceutical composition according to any of clauses 19-20, wherein the dopamine 1 receptor antagonist is SCH23390.

22. The pharmaceutical composition according to any of clauses 19-21, wherein the pharmaceutical composition is formulated for oral, intravenous, intraarterial, intrathecal, intramuscular, subcutaneous, buccal, sublingual, nasal, inhalation, or transdermal administration.

23. The pharmaceutical composition according to any of clauses 19-22, wherein the pharmaceutical composition is formulated for intravenous injection or infusion.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior. may in practice embody something less than exact.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method of reducing the risk of a cocaine-addicted subject relapsing in addiction, the method comprising administering to the subject a combination comprising:
an effective amount of h2E2 monoclonal antibody; and
an effective amount of a dopamine 1 receptor antagonist.

2. The method according to claim 1, wherein the dopamine 1 receptor antagonist is SCH23390.

3. The method according to claim 1, wherein the combination raises the subject's cocaine compulsion zone, thereby reducing drug-seeking behavior of the subject.

4. The method according to claim 1, wherein the h2E2 monoclonal antibody and the dopamine 1 receptor antagonist are administered concurrently or sequentially.

5. The method according to claim 1, wherein administering comprises oral, intravenous, intraarterial, intrathecal, intramuscular, subcutaneous, buccal, sublingual, nasal, inhalation, and transdermal administration.

6. The method according to claim 5, wherein administering comprises intravenous administration of h2E2 and intravenous or inhalation administration of the dopamine 1 receptor antagonist.

7. The method according to claim 1, wherein the h2E2 monoclonal antibody is administered in an amount from about 4 mg/kg to about 400 mg/kg.

8. The method according to claim 1, wherein the dopamine 1 receptor antagonist is administered in an amount from about 0.5 mg to about 100 mg.

9. The method according to claim 1, wherein the subject is a mammal.

10. The method according to claim 9, wherein the subject is a human, a mouse, or a rat.

11. A method of reducing cocaine drug-seeking behavior in a subject in need thereof, the method comprising administering to the subject a combination comprising:
an effective amount of h2E2 monoclonal antibody; and
an effective amount of a dopamine 1 receptor antagonist.

12. The method according to claim 11, wherein the dopamine 1 receptor antagonist is SCH23390.

13. The method according to claim 11, wherein the subject is a mammal.

14. The method according to claim 13, wherein the subject is a human, a mouse, or a rat.

15. A method of reducing the neuromodulatory effect of cocaine on a brain of a subject in need thereof, the method comprising administering to the subject a combination comprising:
an effective amount of h2E2 monoclonal antibody; and
an effective amount of a dopamine 1 receptor antagonist.

16. The method according to claim 15, wherein the dopamine 1 receptor antagonist is SCH23390.

17. The method according to claim 15, wherein the subject is a mammal.

18. The method according to claim 17, wherein the subject is a human, a mouse, or a rat.

19-23. (canceled)
